(19) United States
(12) Patent Application Publication
(10) Pub. No.: US 2017/0173314 A1
(43) Pub. Date: Jun. 22, 2017

(54) NASAL APPLICATOR

(71) Applicant: Shanna NELSON, Oxnard, CA (US)

(72) Inventor: Shanna NELSON, Oxnard, CA (US)

(21) Appl. No.: 14/972,762

(22) Filed: Dec. 17, 2015

(51) Int. Cl. A61M 31/00; A61M 39/20

(52) U.S. Cl. CPC A61M 31/00; A61M 39/20

(57) ABSTRACT

A nasal applicator tip for introducing a viscous medicament along internal nasal walls is provided. The nasal applicator tip may include an elongated tube extending from a base end to a socket end, wherein the socket end forms an enclosure having an opening, and wherein the base end is adapted to removably and fluidly connect to a source of viscous material. An applicator ball may be rotatably housed in the enclosure so as to protrude from the opening and allow viscous medicament to flow between its circumference and the enclosure. A user of the nasal applicator tip may removably attach it to a desired source of viscous material so that the user may selectively engage the protruding portion of the applicator ball and a predetermined portion of the nasal wall for medicating and/or moistening purposes.

### BACKGROUND OF THE INVENTION

The present invention relates to nasal delivery devices and, more particularly, to a nasal applicator for introducing a viscous medicament along internal nasal walls.

In low humidity and dry areas or when a person has a cold or allergies, their nasal passages become dry, sore, crusty, and sensitive. Lip balm is too large to fit inside the nose and is unhygienic to use between the mouth and nose. Nasal rinses help to cleanse the passages of debris, however, nasal rinses leave the nose feeling chapped and irritated as they rinse the nose of its natural oils and moisture. As a result, applying any sort of balm to the inside of the nose required using a q-tip or finger, which tends to be makeshift and messy.

As can be seen, there is a need for a nasal applicator for introducing a viscous medicament into the nose without a mess, thereby reducing the spread of germs through the use of hands and fingers.

### SUMMARY OF THE INVENTION

In one aspect of the present invention, nasal applicator tip for introducing a viscous medicament along internal nasal walls includes an elongated tube extending from a base end to a socket end, wherein the socket end forms an enclosure having an opening, and wherein the base end is adapted to removably and fluidly connect to a source of viscous material; and an applicator ball rotatably housed in the enclosure so as to protrude from the opening, whereby the elongated tube and the applicator ball are adapted to fit into a human nostril.

These and other features, aspects and advantages of the present invention will become better understood with reference to the following drawings, description and claims.

### BRIEF DESCRIPTION OF THE DRAWINGS

FIG. 1 is a top view of an exemplary embodiment of the present invention, shown in use;

FIG. 2 is a perspective view of an exemplary embodiment of the present invention;

FIG. 3 is a perspective view of an exemplary embodiment of the present invention;

FIG. 4 is an exploded view of an exemplary embodiment of the present invention;

FIG. 5 is a section view of an exemplary embodiment of the present invention, taken along line 5-5 in FIG. 2;

FIG. 6 is an enlarged section view of an exemplary embodiment of the present invention, demonstrating movement of an applicator ball;

FIG. 7 is an enlarged section view of an exemplary embodiment of the present invention, demonstrating selective transmission and flow of a viscous material by the applicator ball;

FIG. 8 is a top view of an exemplary embodiment of the present invention;

FIG. 9 is a front view of an exemplary embodiment of the present invention; and

FIG. 10 is a rear view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a nasal applicator tip for introducing a viscous medicament along internal nasal walls. The nasal applicator tip may include an elongated tube extending from a base end to a socket end, wherein the socket end forms an enclosure having an opening, and wherein the base end is adapted to removably and fluidly connect to a source of viscous material. An applicator ball may be rotatably housed in the enclosure so as to protrude from the opening and allow viscous medicament to flow between its circumference and the enclosure. A user of the nasal applicator tip may removably attach it to a desired source of viscous material so that the user may selectively engage the protruding portion of the applicator ball and a predetermined portion of the nasal wall for medicating and/or moistening purposes.

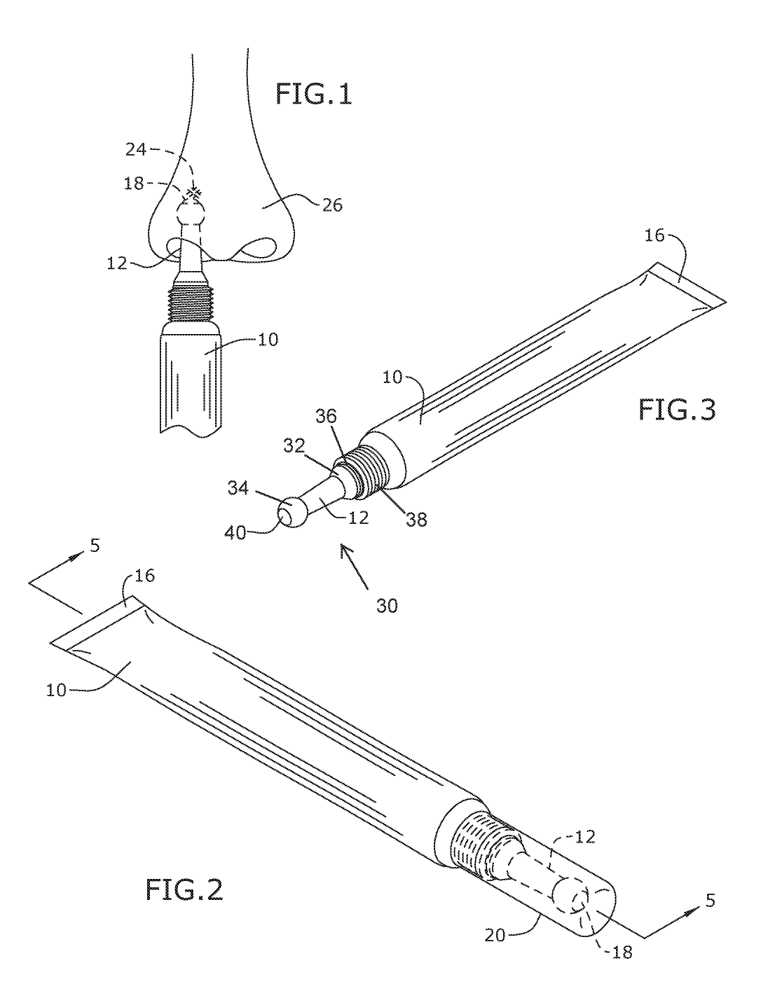

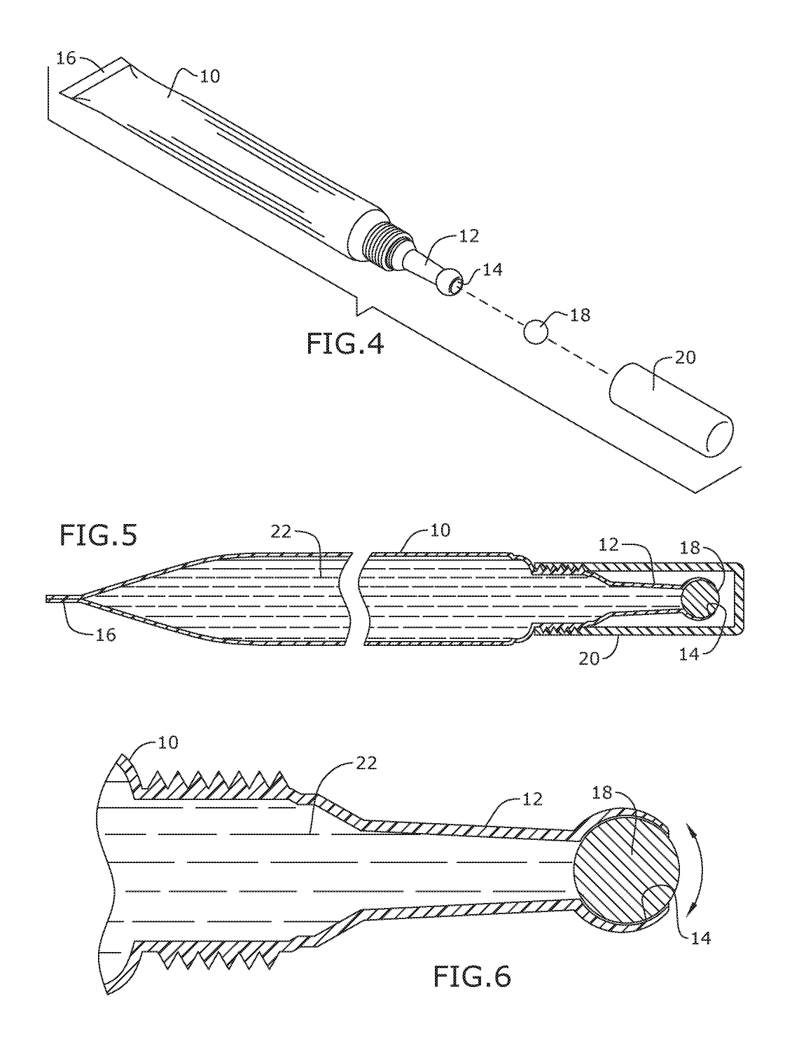

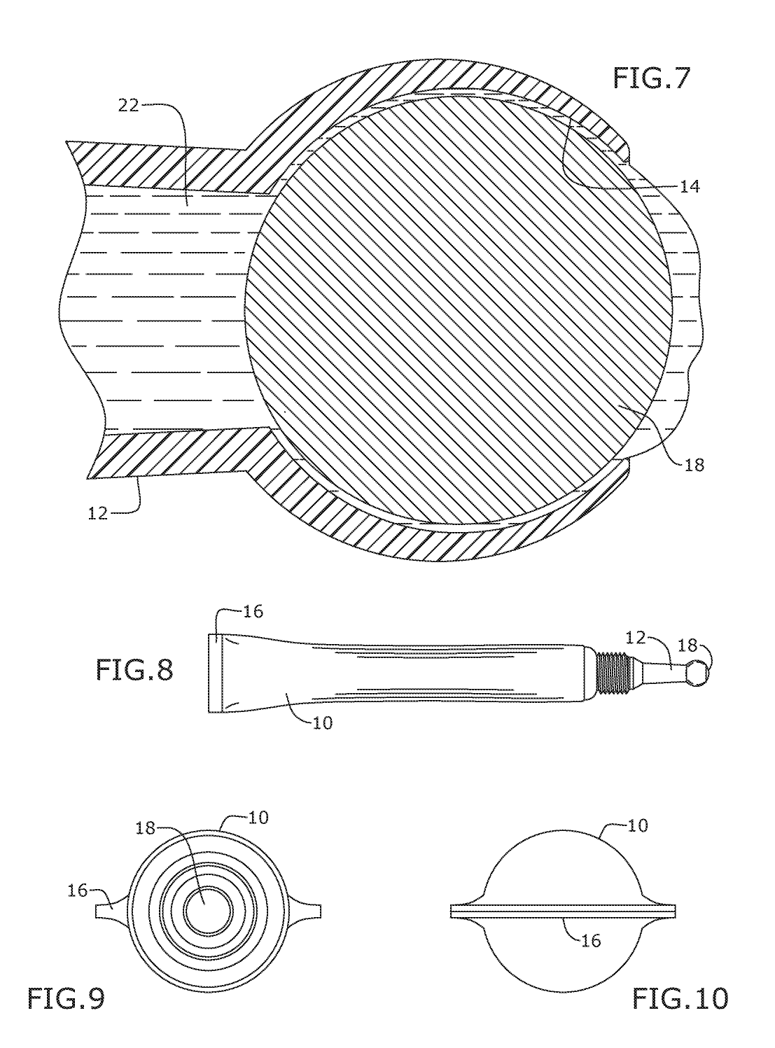

Referring to FIGS. 1 through 10, the present invention may include a nasal applicator tip 30 for introducing a viscous medicament 22 along internal nasal walls 24 of a nose 26. The term internal nasal walls 24 may include walls defining at least an internal portion of the nasal passage and/or nasal cavity, including but not limited to alae walls, nose tip walls, columellar walls, dorsal walls, and septum walls be they anterior, lateral, medial or the like.

The nasal applicator tip 30 may include a base 32, a socket 34, and an elongated tube 12. The elongated tube 12 may fluidly interconnect the base 32 and the socket 34. The base 32 may provide threading 36 for mating with cooperating threading 38 of a squeeze tube 10, thereby the nasal applicator tip 30 may be adapted to be fluidly connected to a variety of squeeze tubes 10 containing the viscous medicament 22. In any event, the nasal applicator tip 30 fluidly connects to the contents of the squeeze tubes 10 so that the viscous medicament 22 may flow through the base 32 and into the elongated tube 12.

The squeeze tubes 10 may provide a pinch area 16 whereby a user may apply pressure to the squeeze tubes 10 thereby regulating the delivery of the viscous medicament 22 through the base 32 and thus through the elongated tube 12 and so into the socket 34.

The present invention may also provide a cap 20 for housing the nasal applicator tip 30, wherein the cap 20 removably connects to a portion of the squeeze tube 10, like the cooperating threading 38.

The socket 34 may form an enclosure 14 having an opening 40. The enclosure 14 may be dimension and adapted to rotatably secure an applicator ball 18 so that the applicator ball 18 is capable of revolving in all directions while a portion of the applicator ball 18 protrudes from the opening 40, as illustrated in FIG. 6. In certain embodiments, the viscous medicament 22 may flow about the applicator ball 18 through a space defined by the enclosure and the circumference of the applicator ball 18. The applicator ball 18 may be any spheroid or semi-spheroid shape as long as it functions in accordance with the present invention as described herein.

The elongated tube 12 is dimensioned to accommodate the length and width of internal nasal walls 24 of the human nose 26. Accordingly, the elongated tube may have a predetermined length to width ratio, wherein the length is defined along a longitudinal axis the elongated tube 12 extends along between the base 32 and the socket 34. The predetermined length to width ratio may be in the range between 3 to 1 to 6 to 1. Likewise, the socket 34 may be dimensioned and adapted to fit within one nostril (nare) of the human nose 26.

In certain embodiments, the squeeze tube 10 and the nasal applicator tip 30 may be a unitary construction connected by a base area 32.

A method of using the present invention may include the following. The nasal applicator tip 30 disclosed above may be provided. A user may affix the nasal applicator tip 30 to a desired squeeze tube 10 so that the viscous medicament 22 contained within the desired squeeze tube 10 may flow through the elongated tube 12 and against the applicator ball 18, whereby a portion of the viscous medicament 22 that contacts portion A of the applicator ball 18 is carried by portion A of the applicator ball 18 as it revolves within the socket 34 until portion A is applied to a predetermined area of the internal nasal walls 24, thereby depositing the portion of the viscous medicament 22 to the predetermined area of the internal nasal walls 24.

The viscous medicament 22 may include a unique mixture of certified organic ingredients forming a balm, ointment or the like. The viscous medicament 22 may be adapted to moisten, heal, and/or shield the predetermined area of the internal nasal walls 24 for a variety of purposes.

It should be understood, of course, that the foregoing relates to exemplary embodiments of the invention and that modifications may be made without departing from the spirit and scope of the invention as set forth in the following claims.

What is claimed is:

1. A nasal applicator tip for introducing a viscous medicament along internal nasal walls, comprising:
an elongated tube extending from a base end to a socket end, wherein the socket end forms an enclosure having an opening, and wherein the base end is adapted to removably and fluidly connect to a source of viscous material; and
an applicator ball rotatably housed in the enclosure so as to protrude from the opening,
whereby the elongated tube and the applicator ball are adapted to fit into a human nostril.

2. The nasal applicator tip of claim 1, further comprising a threading provided along a periphery of the base end.

3. The nasal applicator tip of claim 1, wherein the applicator ball is spheroid.

4. The nasal applicator tip of claim 1, wherein the viscous material comprises an organic medicament.

5. The nasal applicator tip of claim 1, wherein the source of viscous material comprises a squeeze tube.

6. The nasal applicator tip of claim 2, wherein the source of viscous material comprises a squeeze tube.

7. The nasal applicator tip of claim 6, wherein the squeeze tube provides a cooperating threading for mating with the threading.

8. The nasal applicator tip of claim 7, wherein the squeeze tube provides a cap that removably attached to the squeeze tube so as to house the nasal applicator tip.
